(19) United States
(12) Patent Application Publication
(10) Pub. No.: US 2009/0287017 A1
(43) Pub. Date: Nov. 19, 2009

(54) RECOVERY OF AROMATIC DICARBOXLYIC ACIDS FROM WASTE POLYESTER RESIN

(76) Inventors: Hussain Al Ghatta, Fiuggi (Frosinone) (IT); Milan Hronec, Bratislava (SK); James David Richardson, Pagosa Springs, CO (US)

Correspondence Address: EDWIN A. SISSON , ATTORNEY AT LAW , LLC, P.O. BOX 603, SHARON CENTER, OH, 44274-0603, US

(21) Appl. No.: 12/305,451

(22) PCT Filed: Jun. 21, 2006
(86) PCT No.: PCT/IT2006/000475

§ 371 Date: Dec. 18, 2008

(51) Int. Cl. C07C 51/09

(52) U.S. Cl. 562/485

(57) ABSTRACT

The present invention relates to a process of recovering aromatic dicarboxylic acids with low metal contaminants from manufactured articles and/or articles contaminated with PVC or chlorinated compounds, made of or containing aromatic polyester resins, such as beverage bottles, fibers and films, or from waste from processing of these resins. In particular, the invention relates to the recovery of terephthalic acid, isophthalic acid and 2,6-naphthalene dicarboxylic acid from bottles for recycling which are made of PET, PEN or aromatic polyester copolymers.

### BACKGROUND ART

Producers of polyethylene terephthalate have already developed processes for recycling polyethylene terephthalate (PET) scrap from polyester film, fiber and bottle production. The methods for recovering terephthalic acid and ethylene glycol from PET are based on depolymerization, for example by hydrolysis under conditions of neutral pH or in the presence of acids or bases, by acetolysis, methanolysis or glycolysis. U.S. Pat. No. 6,670,503 describes a method of recovering terephthalic acid from PET in the absence of water, with a reagent consisting of one or more metal salts of a weaker acid than the terephthalic acid, until a water-soluble compound is obtained, then subsequently carrying out dissolution in water and acidification.

According to U.S. Pat. No. 6,239,310, PET is heated in an aqueous solution at temperatures from 150° C. to 280° C. with a reagent substance chosen from the group comprising bicarbonates of ammonia and alkali metals, ammonium carbamate and urea. In U.S. Pat. No. 6,545,061, a process is disclosed for depolymerizing and purifying recyclable PET comprising acetolysis to form terephthalic acid and ethylene glycol diacetate.

U.S. Pat. No. 6,562,877 claims a process for depolymerizing recycled colored and contaminated aromatic PET articles with acceptable color characteristics, by depolymerizing said recycled articles in acetic acid at high temperatures (160-250° C.).

U.S. Pat. No. 6,723,873 discloses a process for recovering terephthalic acid from PET by ammoniolysis. In this process, PET is reacted with ammonium hydroxide to form diammonium terephthalate which is then converted to terephthalic acid by heating at a temperature from about 225° C. to about 300° C. Depolymerization of PET by hydrolysis at a high temperature and pressure in the absence of a base or acid, is known, see for example U.S. Pat. No. 4,587,502, or U.S. Pat. No. 4,605,762.

Although various processes are available for hydrolyzing PET waste, the purification of recovered terephthalic acid typically requires several process steps to remove dyes, pigments and other impurities including inorganic compounds and salts. In addition, recrystallization and hydrogenation over noble metal catalysts are usually used to purify terephthalic acid.

Starting materials for making terephthalic acid include such things as mixtures of polyester film, fiber and PET bottle waste. This raw material can also contain metals, labels, lighter plastics, glass, rock and other heavy impurities. These contaminants are removed by such pretreatment processesas skimming or decantation. Depending on the types of contaminants present, bulk solids can also be separated after the hydrolysis process. In a hydrolysis process, PET scrap ground into small particles is depolymerized at temperatures ranging from about 230° C. to about 300° C. and usually at pressures sufficient to maintain a liquid phase.

Recycled PET waste used as feed to the hydrolysis process often contains traces of corrosion product, e.g. PVC (50-200 ppm), metal chlorides, or halogen containing compounds which at the temperature of PET hydrolysis decompose to very corrosive chlorine and/or halogen compounds which attack the reactor. Since the PVC contaminants of the recycled articles can not be excluded, the hydrolytic processes must use Ti equipments to avoid corrosion and high metal content and discoloring of the produced terephthalic acid.

### SUMMARY OF THE INVENTION

This specification discloses a method of recovering aromatic dicarboxylic acids from aromatic polyester resins containing less than 2500 ppm of halogen containing compounds, comprising the step of reacting the aromatic polyester resin in the presence of water at a temperatures in the range of 230° C. to 300° C. wherein the reaction occurs in the presence of 1.0 to 10 wt % of an amine or mixture of amines wherein at least one amine is selected from the group consisting of ammonia, aliphatic amines, aliphatic aromatic amines, and aromatic amines. It is further disclosed that the reaction may proceed in the presence of 1 to 8 wt % of activated charcoal calculated to the amount of the polyester resins. It is also disclosed that the reaction temperature may at maximal 50° C. lower than is reaction temperature and passed through a fixed-bed adsorber filled with activated charcoal. It is further disclosed that the ratio of the amount of water at the end of depolymerization to the amount of aromatic polyester resin present in the reaction mixture is between 4:1 to 10:1 and that the amount the water at the beginning of the reaction may optionally contain ethylene glycol at an amount less than or equal to 10 weight percent with respect to the water plus ethylene glycol mixture. It is further disclosed that the aromatic dicarboxylic acid be separated from the reaction mixture cooled to the temperature in the range from 180 to 90° C.

### DETAILED DESCRIPTION AND DISCLOSURE

This specification describes how to produce terephthalic acid from polyester resin, wherein the metal contents of the resulting terephthalic acid has are below 10 ppm. This process uses a stainless steel reaction and a neutral hydrolytic depolymerization of polyester resin, in particular PET waste, wherein the polyester resin contains less than 2,500 ppm of halogen containing compounds and the hydrolytic depolymerization occurs in the presence of 0.05 to 1 wt % of ammonia and/or aliphatic and/or aromatic amines and polyamines.

The present invention relates to a process of recovering aromatic dicarboxylic acids with low metal contaminants from manufactured articles and/or articles contaminated with PVC or chlorinated compounds, made of or containing aromatic polyester resins, such as beverage bottles, fibers and films, or from waste from processing of these resins.

In particular, the invention relates to the recovery of terephthalic acid, isophthalic acid and 2,6-naphthalene dicarboxylic acid from bottles for recycling which are made of PET, PEN or aromatic polyester copolymers.

Material waste such as PET is broken into chips or fragments that may be produced from mixtures of articles of different colors and different origin. It is also possible to use articles made of or containing polyester resin mixed with polyamides for example, poly (m-xylylene adipamide) or other polymers, particularly those in the form of multi-layer films or multi-layer bottles in which at least one layer is made of co(polyalkylene terephthalate and one layer is made of poly(m-xylylene adipamide), or mixtures thereof with polyalkylene terephthalate. During hydrolytic depolymerization, the polyamides are destroyed to low molecular weight compounds that do not deteriorate the recovered terephthalic acid.

The hydrolytic depolymerization of PET waste proceeds usually in one step at temperatures between 230° C. and 300° C., preferably at 260-275° C. and at a pressure sufficient to maintain liquid phase. To be hydrolytic, there must be some water present in the liquid phase at the start of the reaction. Preferably the amount of water is at least the stoichiometric amount required to recover the theoretical amount of the acid which is 2.0 moles of water per mole of PET.

The term PET waste refers to polyester comprised of terephthalic moieties which can be hydrolytically depolymerized. The polyester may contain other acids or glycols such as isophthalic acid and 2,6 naphthalene dicarboxylic acid. The polyester may also contain other glycols such as diethylene glycol and cyclohexane dimethanol.

Although the examples refer to the recovery of terephthalic acid using a stainless steel vessel, it should be apparent to one of skill in the art that the invention is also suitable to recover 2,6 naphthalene dicarboxylic acid (NDA) from polyethylene naphthalate (PEN).

To avoid the purification of recovered terephthalic acid the hydrolytic depolymerization of PET is performed in the presence of activated charcoal (1-7 wt % to PET) or the reaction mixture after the depolymerization reaction is passed through a fixed-bed adsorber filled with granulated activated charcoal.

The disclosed process is for recovering the acid moiety in the acid form from aromatic polyesters used in film, fibre, bottles, manufacturing residues and other manufactured articles, including reactor purgings, pellets, preforms, and the like. When charged into the depolymerization vessel, the charge should contain less than 2,500 ppm of corrosion compounds, e.g. PVC, metal chlorides, halogenated compounds.

Ammonia, aliphatic/aromatic amine or polyamines containing 1 up to 10 carbon atoms in the molecules are added to the hydrolytic process to suppress the corrosion properties of halogen containing compounds present in the PET waste. The C₁-C₁₀ aliphatic amines, C₂, C₄, C₆ and C₈ aliphatic diamines, hexamethylene triamine, benzylamine, xylylene diamines are all believed to be very efficient. They are dosed to the reaction system at the beginning and/or during the depolymerization reaction in amounts ranging from 0.05 to 1 wt % calculated to polyester resin.

The reaction is carried out in a stainless steel reactor for 30-120 minutes and can be performed continuously or discontinuously. The hydrolytic depolymerization proceeds in water, mixtures of ethylene glycol and water (up to 10 wt %), or steam which is purged into the reaction system. The weight ratio of PET/water is from 1:4 to 1:10, preferably 1:5 to 1:7. The depolymerization process can be carried out in air or inert gas, which is most preferred. The produced terephthalic acid is separated from the liquid phase by conventional methods, for example after cooling by filtration.

Terephthalic acid prepared according to the present invention does not contain polyamides or amino compounds in detectable amounts. The iron content is below 20 ppm which is indicative of the non-corrosive nature of the process. The L* color parameters of recovered terephthalic acid prepared, even those from the mixture of green, blue and brown colored PET waste, are above 82-86.

The following examples illustrate but do not limit the scope of the present invention.

### Example 1

27 g of PET, 150 g of deionized water and 0.040 g of PVC (1,500 ppm on PET) were weighed into 250 ml stainless steel rocking autoclave. Inside the reactor, a basket was filled with 0.5 g of granulated (0.6-0.7 mm) activated charcoal. The reactor was twice purged with nitrogen and heated at 270° C. for 60 minutes. Terephthalic acid isolated by filtration, washed with ca. 75° C. deionized water (3×50 ml) and dried at 95° C. overnight contained 43.4 ppm of iron.

### Example 2

The procedure of Example 1 was repeated except that 0.13 g of hexamethylenediamine was added. The recovered terephthalic acid contained 15.4 ppm of iron.

### Example 3

The procedure of Example 1 was repeated except that 0.150 g t-octylamine was added. The recovered terephthalic acid contained 16.9 ppm of iron. The content of nitrogen is below 0.1 ppm and the color parameters are L*=84.3, a*=−1.09 and b*=2.97.

### Example 4

The procedure of Example 1 was repeated except that 0.150 g of dibenzylamine was added. The recovered terephthalic acid contained 18.1 ppm of iron.

### Example 5

The procedure of Example 1 was repeated except that 0.150 g of diethylenetriamine was added. The recovered terephthalic acid contained 17.9 ppm of iron.

### Example 6

The procedure of Example 1 was repeated except that 0.300 g of 25% aqueous solution of ammonia and 150 g of water containing 4.1 wt % of ethylene glycol was added. The recovered terephthalic acid contained 19.0 ppm of iron.

### Example 7

The procedure of Example 1 was repeated except that 0.150 g of m-xylylenediamine was added. The recovered terephthalic acid contained 19.9 ppm of iron.

1. A method of recovering aromatic dicarboxylic acids from aromatic polyester resins containing less than 2,500 ppm of halogen containing compounds, comprising the step of depolymerizing the aromatic polyester resin with water at a temperature in the range of 230° C. to 300° C. wherein the depolymerization reaction occurs in the presence of at least one amine selected from the group consisting of ammonia, aliphatic amines, aliphatic aromatic amines, and aromatic amines.

2. A method according to claim 1 wherein the amine is present in the range of 0.05 to 1.0 weight percent of the aromatic polyester resins in the reaction.

3. A method according to claim 1, in which the depolymerization proceeds in the presence of 1 to 8 wt % of activated charcoal calculated to the amount of polyester resin.

4. A method according to claim 1 in which the reaction mixture is passed through a fixed-bed adsorber filled with activated charcoal at a temperature in the range of 50° C. below the depolymerization temperature of the polyester with water and the depolymerization temperature of the polyester with water.

5. A method according to claim 4, in which the ratio of the amount of water at the end of the depolymerization to the amount of aromatic polyester resin present in the reaction mixture is between 4:1 to 10:1.

6. A method according to claim 5 in which the water at the beginning of the reaction contains ethylene glycol at an amount less than or equal to 10 weight percent with respect to the water plus ethylene glycol mixture.

7. A method according to claim 6 wherein the aromatic dicarboxylic acid is separated from the reaction mixture cooled to a temperature in the range from 180 to 90° C.

8. A method according to claim 2, in which the depolymerization proceeds in the presence of 1 to 8 wt % of activated charcoal calculated to the amount of polyester resin.

9. A method according to claim 2 in which the reaction mixture is passed through a fixed-bed adsorber filled with activated charcoal at a temperature in the range of 50° C. below the depolymerization temperature of the polyester with water and the depolymerization temperature of the polyester with water.

10. A method according to claim 9, in which the ratio of the amount of water at the end of the depolymerization to the amount of aromatic polyester resin present in the reaction mixture is between 4:1 to 10:1.

11. A method according to claim 10 in which the water at the beginning of the reaction contains ethylene glycol at an amount less than or equal to 10 weight percent with respect to the water plus ethylene glycol mixture.

12. A method according to claim 11 wherein the aromatic dicarboxylic acid is separated from the reaction mixture cooled to a temperature in the range from 180 to 90° C.
